(19) United States
(12) Patent Application Publication
(10) Pub. No.: US 2025/0381164 A1
(43) Pub. Date: Dec. 18, 2025

(54) COMPOSITIONS AND METHODS OF USE FOR THE TREATMENT OF DIABETES

(71) Applicant: Jesus ZARAZUA-GARCIA, Wichita, KS (US)

(72) Inventor: Jesus ZARAZUA-GARCIA, Wichita, KS (US)

(21) Appl. No.: 18/880,167

(22) PCT Filed: Jun. 29, 2023
(86) PCT No.: PCT/US2023/026624

§ 371 Date: Dec. 30, 2024

Related U.S. Application Data
(60) Provisional application No. 63/367,539, filed on Jul. 1, 2022.

(51) Int. Cl. A61K 31/28; A61K 31/519; A61K 31/714; A61K 38/17; A61P 3/10

(52) U.S. Cl. CPC A61K 31/28; A61K 31/519; A61K 31/714; A61K 38/1709; A61P 3/10

(57) ABSTRACT

Compositions and methods of use for the treatment of diabetes are provided. The compositions comprise key compounds including chromium, folate, vitamin B12, and intrinsic factor.

### CROSS-REFERENCE TO RELATED APPLICATION(S)

This application claims priority to U.S. Provisional Application Ser. No. 63/367,539 filed Jul. 1, 2022, the disclosure of which is hereby incorporated by reference in its entirety.

### FIELD OF THE INVENTION

Compositions and methods of use for the treatment of diabetes have been developed. The compositions comprise key compounds including chromium picolinate, folate, vitamin B12, and intrinsic factor

### BACKGROUND OF THE INVENTION

Diabetes is a metabolic disorder characterized by high blood sugar over a long period of time. It is divided into two types, type I and type II. Type I diabetes is the result of the pancreas not producing enough insulin due to the loss of beta cells, while type II diabetes is the result of insulin resistance.

Diabetes affects hundreds of millions of people worldwide and represents a significant healthcare burden. Short term complications of diabetes include diabetic ketoacidosis and hyperosmolar hyperglycemic state. Long term complications of diabetes include cardiovascular disease, stroke, chronic kidney disease, foot ulcers or amputation, and damage to nerves. eyes. and cognition.

Current treatments involve monitoring of blood sugar, improved diet and exercise, and administration of a variety of prescription of drugs and insulin. Monitoring of blood sugar and administration of insulin can require multiple needle pricks a day, which is both expensive and unpleasant for the patient. Similarly, prescription drugs can be expensive and difficult to obtain access. Thus, there remains a need for inexpensive and accessible diabetes treatments.

### BRIEF SUMMARY OF THE INVENTION

Disclosed herein is a pharmaceutical composition comprising chromium picolinate. folate. vitamin B12, and intrinsic factor.

Various methods are disclosed herein including a method for treating type I or type II diabetes. The method comprises administering any one of the pharmaceutical compositions described herein to a subject in need thereof.

Other objects and features will be in part apparent and in part pointed out hereinafter.

### DETAILED DESCRIPTION OF THE INVENTION

Having described the invention in detail, it will be apparent that modifications and variations are possible without departing from the scope of the invention defined in the appended claims.

This disclosure is directed to a pharmaceutical composition including chromium. folate. vitamin B12, and intrinsic factor. The composition includes chromium in an administered dosage ranging from about 35 mcg to about 1,500 meg, about 200 meg to about 1,500 mcg, about 500 mcg to about 1,500 meg. or about 900 meg to about 1,100 mcg. Folate is present in the composition in a dosage ranging from about 100 meg to about 2,000 mcg, about 400 mcg to about 1,500 mcg. or about 1. 100 meg to about 1,400 meg. The composition includes vitamin B12 in a dosage of from about 5 meg to about 2,000 mcg, about 50 meg to about 1,000 meg, or about 400 meg to about 600 meg. The composition includes intrinsic factor in a dosage ranging from about 0.5 mg to about 100 mg, about 1 mg to about 50 mg, or about 15 mg to about 25 mg. In one embodiment, the composition suitably includes at least 1.000 meg chromium picolinate, at least 1,330 mcg folate, at least 500 mcg vitamin B12, and at least 20 mg intrinsic factor.

The components of the composition can be present in several different forms. The chromium can be in the form of elemental chromium, chromium picolinate, chromium nicotinate. chromium polynicotinate, chromium chloride. and chromium histidinate. In one particularly suitable embodiment, the chromium is in the form of chromium picolinate. The folate can be in the form of tetrahydrofolate, 5-methyl-tetrahydrofolate, calcium L-5-methyltetrahydrofolate, (6S)-5-methyltetrahydrofolic acid glucosamine salt, or folic acid. Suitably, the folate is in the form of calcium L-5-methyltetrahydrofolate. The vitamin B12 can be in the form of methylcobalamin, adenosylcobalamin, hydroxycobalamin, cyanocobalamin. Suitably, vitamin B12 is in the form of methylcobalamin. Intrinsic factor is also known as vitamin B12 intrinsic factor. It is a glycoprotein that helps the body absorb vitamin B12.

The composition can be formulated as one or more capsules or tablets. The composition can further include at least one pharmaceutically acceptable excipient or adjuvant. The excipient can be selected from the group consisting of dicalcium phosphate, magnesium sterate, silica. croscarmellose. microcrystalline cellulose, cellulose. stearic acid, croscarmellose sodium, and any other well-known filler in the art.

Alternatively, the composition can be formulated as an intramuscular injection. The composition can further comprise at least one pharmaceutically acceptable excipient or adjuvant. The excipient can comprise a fluid such as saline or any other well-known carrier in the art.

This disclosure is also directed to a method of treating type I or type II diabetes, wherein the method comprises administering any one of the pharmaceutical compositions described herein to a subject in need thereof.

The method can include administering the pharmaceutical composition at least once a week or at least once daily to the subject. In one suitable embodiment, the composition is administered once daily to the subject. The subject is preferably a human.

The method can further include administering at least one additional therapeutic agent for the treatment of type I or type II diabetes. The at least one additional therapeutic agent can be selected from the group consisting of insulin, an amylinomimetic drug (pramlintide, etc.), an alpha-glucosidase inhibitor (acarbose, miglitol, voglibose, etc.), a biguanide (metformin, phenformin, buformin, etc.), a dopamine agonist (bromocriptine, etc.), a dipeptidyl peptidase-4 (DPP-4) inhibitor (alogliptin, gemigliptin, linagliptin, saxagliptin, sitagliptin, teneligliptin, vildagliptin, etc.), a glucagon-like peptide-1 (GLP-1) receptor agonist (albiglutide, dulaglutide, exenatide, liraglutide, lixisenatide, semaglutide, taspoglutide. etc.), a Lyn kinase activator (tolimidone, etc.) a meglitinde (nateglinide, repaglinide, etc.), a sodium-glucose transporter (SGLT) 2 inhibitor (dapaglifloxin, canagliflozin, empagliflozin, ertugliflozin, remogliflozin, etc.), a sulfonylurea (glimepiride, glipizide, gliclazide, glyburide, glibenclamide, glyclopyramide, gliquidone, acetohexamide, chloropropamide. tolazamide, tolbutamide. etc.), and a thiazolidinedione (rosiglitazone, pioglitazone, troglitazone, etc.)

The method can be for treating type I or type II diabetes. The method preferably results in controlled blood sugar levels in the subject. The method can result in fasting glucose levels <6.1 mmol/L and HbA_{1c} levels of <42 mmol/mol. The method of administering a pharmaceutical composition comprising chromium picolinate, folate, vitamin B12. and intrinsic factor can also result in reducing the need for an additional therapeutic agent to treat diabetes in the subject, such as reducing or eliminating the subject's need for insulin administration.

### EXAMPLES

The following non-limiting examples are provided to further illustrate the present invention.

### Example 1: Diabetes Treatment Case Study

A male patient had type I and type II diabetes for about a year and 10 months. The patient took daily supplements including 1,000 mcg chromium (chromium picolinate), 1,330 meg folate (calcium L-5-methyltetrahydrofolate), 500 mcg vitamin B12 (methylcobalamin), and 20 mg intrinsic factor. The patient was eventually informed by his doctor's office that he no longer needed to take insulin.

When introducing elements of the present invention or the preferred embodiments(s) thereof, the articles “a”, “an”, “the” and “said” are intended to mean that there are one or more of the elements. The terms “comprising”, “including” and “having” are intended to be inclusive and mean that there may be additional elements other than the listed elements.

In view of the above, it will be seen that the several objects of the invention are achieved and other advantageous results attained.

As various changes could be made in the above compositions and processes without departing from the scope of the invention, it is intended that all matter contained in the above description shall be interpreted as illustrative and not in a limiting sense.

1. A pharmaceutical composition comprising chromium, folate, vitamin B12, and intrinsic factor.

2. The pharmaceutical composition of claim 1, wherein the composition comprises at least 1,000 mcg chromium, at least 1,330 mcg folate, at least 500 meg vitamin B12, and at least 20 mg intrinsic factor.

3. The pharmaceutical composition of claim 1, wherein the folate is in the form of calcium L-5-methyltetrahydrofolate.

4. The pharmaceutical composition of claim 1, wherein the vitamin B12 is in the form of methylcobalamin.

5. The pharmaceutical composition of claim 1, wherein the chromium is in the form of chromium picolinate.

6. The pharmaceutical composition of claim 1, wherein the composition comprises at least one pharmaceutically acceptable excipient or adjuvant.

7. The pharmaceutical composition of claim 6, wherein the excipient is selected from the group consisting of dicalcium phosphate, magnesium sterate, silica, croscarmellose, microcrystalline cellulose, cellulose, stearic acid, and croscarmellose sodium.

8. The pharmaceutical composition of claim 1, wherein the composition is formulated as one or more capsules or tablets.

9. The pharmaceutical composition of claim 1, wherein the composition is formulated as an injection.

10. A method of treating type I or type II diabetes, the method comprising administering a pharmaceutical composition comprising chromium, folate, vitamin B12, and intrinsic factor to a subject in need thereof.

11. The method of claim 10, wherein the method comprises administering the pharmaceutical composition once daily to the subject in need thereof.

12. The method of claim 10 claim 10, wherein the subject is a human.

13. The method of claim 10, wherein the method comprises administering at least one additional therapeutic agent for the treatment of type I or type II diabetes.

14. The method of claim 10, wherein the method is for treating type I diabetes.

15. The method of claim 10, wherein the method is for treating type II diabetes.

16. The method of claim 10, wherein the method results in controlled blood sugar levels in the subject.
